(19) United States
(12) Patent Application Publication
(10) Pub. No.: US 2017/0165389 A1
(43) Pub. Date: Jun. 15, 2017

(54) AUTOMATED HEAT TRANSFER PRESS AND METHOD FOR AUTOMATED HEAT TRANSFER PRESSING

(71) Applicant: DIAMOND PRINT INC., Commerce, CA (US)

(72) Inventor: TA WEI CHAO, Commerce, CA (US)

(21) Appl. No.: 14/964,468

(22) Filed: Dec. 9, 2015

(51) Int. Cl. B41M 5/382; H02P 25/

(52) U.S. Cl. CPC A61L 9/03

(57) ABSTRACT

An automated heat transfer press and method for automated heat transfer pressing provides an electric linear actuator motor that induces an arced translation of a first plate onto a second plate for dye sublimation printing. The arced motion of the first plate enables the first plate to press against the second plate at an angle, rather than a vertical pressing action; whereby adaption to a variously sized and dimensioned substrates is possible. A second link bar adjusts alignment of the second plate with first plate. An electric linear actuator motor manipulates a rod through electrical power. Rod is extended to create linear motion. The rod operatively connects to a hinged arm, applying linear force that causes the hinged arm to displace the first plate at arced motion. A control portion regulates the linear actuator motor to press and release the plates against each other in predetermined temperatures and intervals.

### FIELD OF THE INVENTION

The present invention relates generally to an automated heat transfer press and method for automated heat transfer pressing of an image on a substrate. More so, an automated heat transfer press provides an electric linear actuator motor that induces an arced translation of at least one first plate onto at least one second plate to achieve efficient dye sublimation printing, and also provides a control portion for regulating the linear actuator motor to press and release the plates against each other in predetermined temperatures and durations of time.

### BACKGROUND OF THE INVENTION

Typically, heat transfer presses are configured to apply graphics and photographs to objects such as drinking mugs. This unique form of printing enables personalization of oddly shaped and sized objects at a reasonable cost.

In general, the sublimation process works by transferring permanent, high-temperature sublimation dies containing a desired photograph or print, onto items such as ceramic mugs, tiles, beer steins and the like. The image is first transferred to a paper mat containing sublimation dies forming the desired image in the desired colors. Using well known techniques, this mat is pressed onto the object using a combination of heat and pressure by the heat transfer press. This results in a permanent bond between the sublimation dies and the object.

Generally, the sublimation printer employs a printing process that uses heat to transfer dye onto materials such as a plastic, card, paper, or fabric. The sublimation name was first applied because the dye was considered to make the transition between the solid and gas states without going through a liquid stage.

It is also known that sublimation printing is a digital printing technology using full color artwork that works with polyester and polymer-coated substrates. Also referred to as digital sublimation, the process is commonly used for decorating apparel, signs and banners, as well as novelty items such as cell phone covers, plaques, coffee mugs, and other items with sublimation-friendly surfaces. The process uses the science of sublimation, in which heat and pressure are applied to a solid, turning it into a gas through an endothermic reaction without passing through the liquid phase.

However, even among similarly sized coffee cups, size differences occur, for example, among different batches or manufacturing lots. Such size differences may result in variations in the pressure applied by the plates of the heat transfer press to the coffee cup. This can result in too much, or too little, plate pressure which can adversely affect the heat transfer pressing process.

Other proposals have involved sublimation printing. The problem with these devices is that they press down directly on the object, which may be problematic if the object has an odd shape or size. Thus, an unaddressed need exists in the industry to address the aforementioned deficiencies and inadequacies. Even though the above cited heat transfer presses meet some of the needs of the market, a heat transfer press that applies heated pressure on a substrate from an arced motion to enhance the heat transfer pressing process, and also controls the duration, temperature, and pressure is still desired.

### SUMMARY OF THE INVENTION

The present invention is directed to an automated heat transfer press and method for automated heat transfer pressing through a fully electrical and automated process. The press provides automated heat transfer pressing through a heat transfer press that applies heated pressure on a substrate from an arced motion to enhance the heat transfer pressing process. In one embodiment, the automated heat transfer press, hereafter, “press”, provides an electric linear actuator motor that induces an arced translation of at least one heated first plate onto at least one fixed second plate to achieve efficient dye sublimation printing. The press further includes a control portion for regulating the linear actuator motor to press and release the plates against each other in predetermined intervals.

In some embodiments, the press may transfer permanent, high-temperature sublimation dyes containing a desired photograph or print, onto substrates, such as ceramic mugs, tiles, trophies, garments, chips, beer steins, and the like.

The first plate is sufficiently heated (200°-500° Fahrenheit) and pressed against the second plate at a sufficient pressure to achieve sublimation and transfer of an image, i.e. dye, ink, etc, onto the substrate. The substrate may be shaped and dimensioned planar, circular, rectangular, cubical, or pyramidal. The press utilizes a unique arced motion to apply the dye onto the substrate, no matter the shape or size.

In some embodiments, the press may include a heated first plate and a fixed second plate. The first plate may be disposed above the second plate. The first plate is mobile, while the second plate is generally fixed. The first plate may include embedded heating pipes that carry thermal energy uniformly through the plates.

In some embodiments, the first plate may be actuated by an electric linear actuator motor. The electric linear actuator motor utilizes a plurality of gears and a speed control mechanism to manipulate a rod through electrical power. The electrical linear actuator motor is configured to extend and retract the rod in a reciprocating linear motion. The rod operatively connects to a hinged arm, and applies a linear force that causes the hinged arm to displace the first plate in an arced motion. The hinged arm serves as the link between the electric linear actuator motor and the first plate. In this manner, the first plate is induced in an arced translation on and off the second plate.

The arced motion of the first plate enables the first plate to press against the second plate at an angle, rather than a vertical pressing action. The angled engagement between the first and second plates allows the plates to adapt to a variety of sizes of objects, such as drinking mugs, or to adapt to different kinds of objects altogether. For example, even among similarly sized coffee cups, size differences occur, for example, among different batches or manufacturing lots. Such size differences may result in variations in the pressure applied by the plates of the heat transfer press to the coffee cup. This can result in too much, or too little, plate pressure which can adversely affect the heat transfer pressing process.

The press further includes a control portion that automates the linear actuator motor to press and release the plates against each other in predetermined intervals. For example, the first plate is actuated to press onto the second plate every thirty seconds, and then to release before repressing again in fifteen seconds, and so on. The control portion may also be used to regulate the temperature and pressure of the plates, and to power on and off the press. Thus, the control portion reduces the need for manual labor and enables more efficient heat transfer pressing. Both the electric linear actuator motor and the control portion are fully electrical in operation.

In one aspect, an automated heat transfer press for efficient dye sublimation printing, the press comprising:
a frame, the frame configured in a fixed position for supporting the press;
a second plate, the second plate configured to join with the frame;
a first plate, the first plate configured to controllably generate heat, the first plate further configured to reciprocally move in an arced motion towards and away from the second plate,
whereby the first plate is disposed in a spaced-apart relationship to the second plate from an open position,
whereby the first plate is disposed in an engaged, substantially aligned relationship with the second plate from a pressing position;
a second link bar, the second link bar configured to laterally displace the second plate out of alignment with the first plate from the open position, the second link bar further configured to laterally displace the second plate in alignment with the first plate from the pressing position;
an electric linear actuator motor, the electric linear actuator motor comprising a rod, the electric linear actuator motor configured to extend and retract the rod in a linear motion;
a hinged arm, the hinged arm configured to operatively join with the rod of the electric linear actuator motor, the hinged arm further configured to translate the linear motion of the rod to the arced motion;
a plate adapter, the plate adapter configured to operatively connect the hinged arm to the first plate,
whereby the arced motion of the hinged arm translates to the arced motion of the first plate;
a control portion, the control portion configured to regulate actuation of the electric linear actuator motor, the control portion further configured to set a predetermined temperature and a predetermined duration,
whereby the control portion actuates the electric linear actuator motor to induce the first plate to the pressing position when the predetermined temperature has been achieved,
whereby the control portion actuates the electric linear actuator motor to induce the first plate to the open position when the predetermined duration has expired.

In another aspect, the press is a dye sublimation heat press.

In another aspect, the first plate comprises imbedded heated tubes.

In another aspect, the first plate and the second plate are generally rectangular shaped.

In another aspect, the second plate further comprises a pair of draw out slides, the pair of draw out slides configured to expand and retract the surface area of the second plate.

In another aspect, the electric linear actuator motor further comprises a plurality of gears and a speed control mechanism, the plurality of gears and a speed control mechanism configured to control displacement of the rod.

In another aspect, the hinged arm further includes an arm adapter, the arm adapter configured to operatively connect the hinged arm to the electric linear actuator motor.

In another aspect, the hinged arm further includes a first link bar, the first link bar configured to pivot the hinged arm about the frame.

In another aspect, the hinged arm further includes a set of adapter columns, the set of adapter columns configured to hingedly mount the hinged arm to the frame.

In another aspect, the press further includes a housing, the housing configured to at least partially cover the hinged arm and the electric linear actuator motor.

In another aspect, the control portion comprises a digital temperature and time controller, the digital temperature and time controller configured to set a predetermined temperature and a predetermined duration for operation of the press.

In another aspect, the control portion comprises a display screen for indicating temperature, pressure, and time.

In another aspect, the press further includes an electrical control box, the electrical control box configured to regulate electrical components of the press.

In another aspect, the press comprises a start switch, the start switch operatively connected to a power source, the start switch configured to power on and off the press.

In another aspect, the press comprises a limit switch, the limit switch configured to control the speed and pressure of the first plate moving towards the pressing position.

In another aspect, the first plate is configured to induce sublimation on an image for at least partially transferring the image to a substrate positioned on the second plate.

One objective of the present invention is to provide a fully automated, electrical heat transfer press.

Another objective is to provide a fully electric heat transfer press instead of the traditional electric and pneumatic control mode.

Another objective is to provide simplified control procedures, with no compressor; and thereby reducing business investment and saving space.

Another objective is to enable a press operator to release the hands, without requiring manually pressing down; thereby creating less fatigue to the hands and eyes.

Another objective is to provide a quiet heat transfer press by not utilizing a compressor.

Another objective is to provide a heat transfer press in which the position of the heads is easily adjustable to accommodate minor variations in the sizes of the object being sublimated.

Another objective is to provide a heat transfer press which can be adapted to handle different kinds of objects without requiring removal of the first and second plates. It is further desirable that this conversion can be accomplished in a relatively short period of time by a user.

Yet another objective is to provide a heat transfer press which maintains relatively cool exterior temperatures while in operation.

Yet another objective is to provide a heat transfer press which is relatively inexpensive, durable, and requires little maintenance.

Other systems, devices, methods, features, and advantages will be or become apparent to one with skill in the art upon examination of the following drawings and detailed description. It is intended that all such additional systems, methods, features, and advantages be included within this description, be within the scope of the present disclosure, and be protected by the accompanying claims and drawings.

### BRIEF DESCRIPTION OF THE DRAWINGS

The invention will now be described, by way of example, with reference to the accompanying drawings, in which:

FIG. 1 illustrates a blowup view of an exemplary automated heat transfer press, in accordance with an embodiment of the present invention;

FIG. 2 illustrates a perspective view of an automated heat transfer press, in accordance with an embodiment of the present invention;

FIG. 3 illustrates a frontal view of an automated heat transfer press, in accordance with an embodiment of the present invention;

FIG. 4 illustrates an elevated side view of an automated heat transfer press, in accordance with an embodiment of the present invention;

FIG. 5 illustrates a top view of an exemplary automated heat transfer press, in accordance with an embodiment of the present invention;

FIG. 6 illustrates a perspective view of an exemplary electric linear actuator motor, in accordance with an embodiment of the present invention;

FIG. 7 illustrates a perspective view of an exemplary hinged arm, in accordance with an embodiment of the present invention; and

FIG. 8 illustrates a flowchart diagram of an exemplary method for heat transfer pressing, in accordance with an embodiment of the present invention.

Like reference numerals refer to like parts throughout the various views of the drawings.

### DETAILED DESCRIPTION OF THE INVENTION

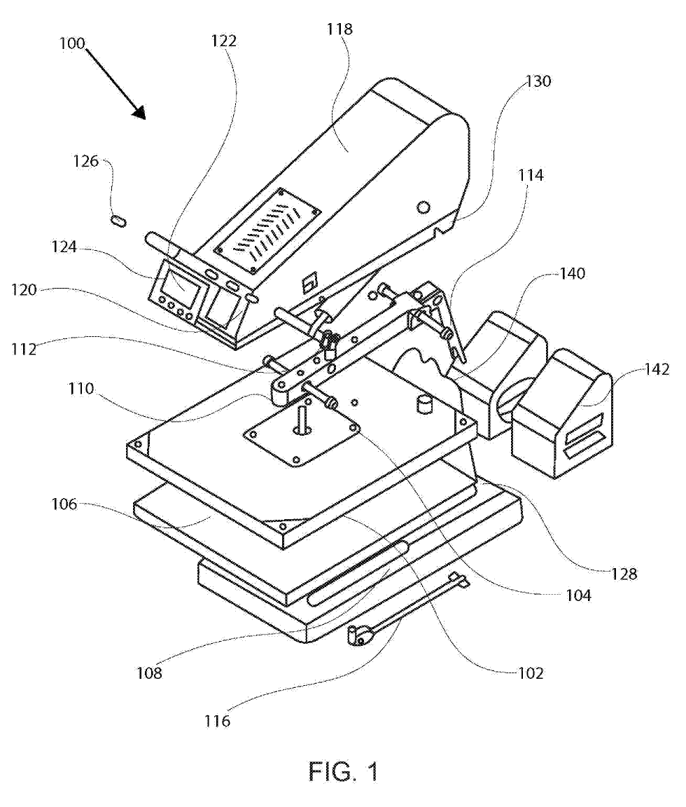

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “first,” “second,” “left,” “rear,” “right,” “front,” “vertical,” “horizontal,” and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

At the outset, it should be clearly understood that like reference numerals are intended to identify the same structural elements, portions, or surfaces consistently throughout the several drawing figures, as may be further described or explained by the entire written specification of which this detailed description is an integral part. The drawings are intended to be read together with the specification and are to be construed as a portion of the entire “written description” of this invention as required by 35 U.S.C. §112.

In one embodiment of an automated heat transfer press 100 and method 200 for automated heat transfer pressing is presented in FIGS. 1-8. The automated heat transfer press 100, hereafter, “press 100” provides automated heat transfer pressing through a fully electrical heat transfer press 100 that applies heated pressure on a substrate from an arced motion to enhance the heat transfer pressing process.

As FIG. 1 references, the press 100 may include an electric linear actuator motor 130 that induces an arced translation of at least one heated first plate 102 onto at least one fixed second plate 106 to achieve efficient dye sublimation printing. The press 100 further provides a control portion 120 for automating the linear actuator motor 130 to press 100 and release the plates 102, 106 against each other in predetermined temperatures and predetermined durations of time.

Those skilled in the art will recognize that the press 100 works to transfer permanent, high-temperature sublimation dyes containing a desired photograph or print, onto substrates, such as ceramic mugs, tiles, trophies, garments, chips, beer steins, and the like. The at least one first plate 102 is sufficiently heated (200°-500° Fahrenheit) and pressed against the at least one second plate 106 at a sufficient pressure to achieve sublimation and transfer of an image, i.e. dye, ink, etc, onto the substrate. The substrate may be shaped and dimensioned planar, circular, rectangular, cubical, or pyramidal. The press utilizes a unique arced motion to apply the dye onto the substrate, no matter the shape or size.

In some embodiments, the press 100 is a sublimation heat press 100. The press 100 is configured to transfer an image from a transfer material and a transfer paper on a substrate positioned on the second plate 106. The image may include a logo, an animated drawings, and text. The substrate may include, without limitation, a coffee mug, a plate, a chip, a garment, and a plastic or metal panel.

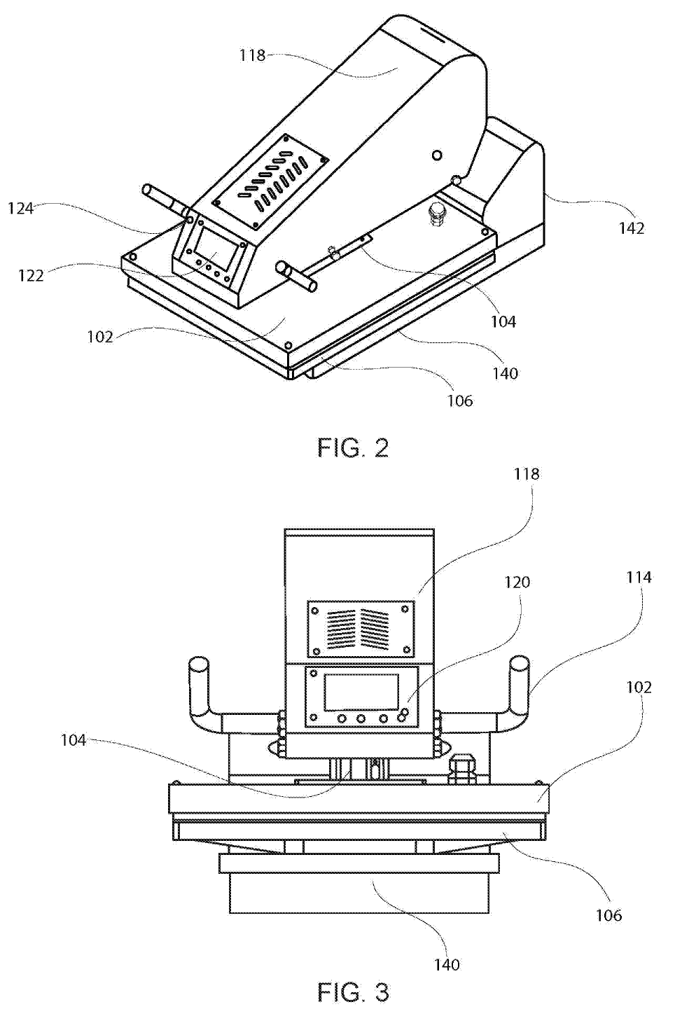

Looking now at FIG. 2, the press 100 may include a housing 118 that at least partially covers the hinged arm 110 and the electric linear actuator motor 130. A pair of arms 144 may extend from the housing 118 for enhanced control of the first plate 102. The housing 118 may be fabricated from a metal or a rigid polymer that can withstand high temperatures without deforming.

The press 100 performs a substantial portion of the heat transfer pressing through a first plate 102 and a second plate 106. The first plate 102 may be disposed above the second plate 106. The first plate 102 is mobile, while the second plate 106 is generally fixed. The first plate 102 may include embedded heating pipes that carry thermal energy uniformly through the core of the first plate 102. The second plate 106 remains fixed to the frame 140. However, the second plate 106 is mobile so as to laterally adjust in and out of alignment with the first plate 102.

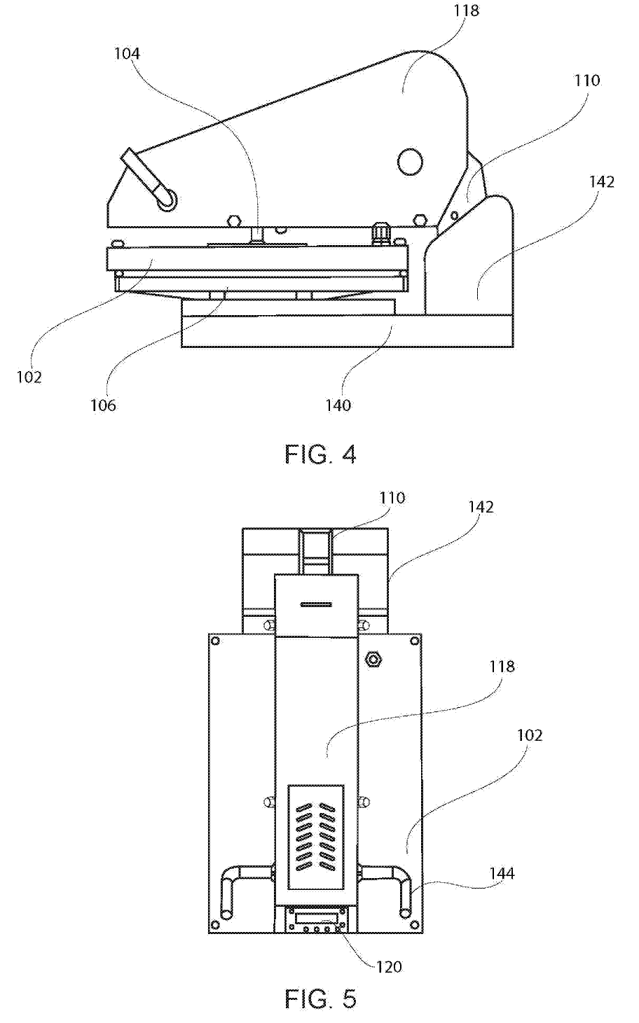

As illustrated in FIG. 3, the first plate 102 is configured to controllably generate heat for sublimation of a dye onto a substrate. The first plate 102 is further configured to reciprocally move in an arced motion towards and away from the second plate 106. Thus, in one embodiment, the first plate 102 is disposed in a spaced-apart relationship to the second plate 106 from an open position. In another embodiment, the first plate 102 is disposed in an engaged, substantially aligned relationship with the second plate 106 from a pressing position (FIG. 4). The pressure applied by the first plate 102 is adjustable through a lii switch 128.

As FIG. 5 illustrates, the second plate 106 comprises a pair of draw out slides 108 configured to expand and retract the surface area of the second plate 106. A second link bar 116 is configured to manipulate the draw out slides 108. The second link bar 116 is further configured to laterally displace the second plate 106 out of alignment with the first plate 102 from the open position. The second link bar 116 is further configured to laterally displace the second plate 106 in alignment with the first plate 102 from the pressing position. Thus, the second plate 106 is synchronized to adjust its alignment based on the position of the first plate 102.

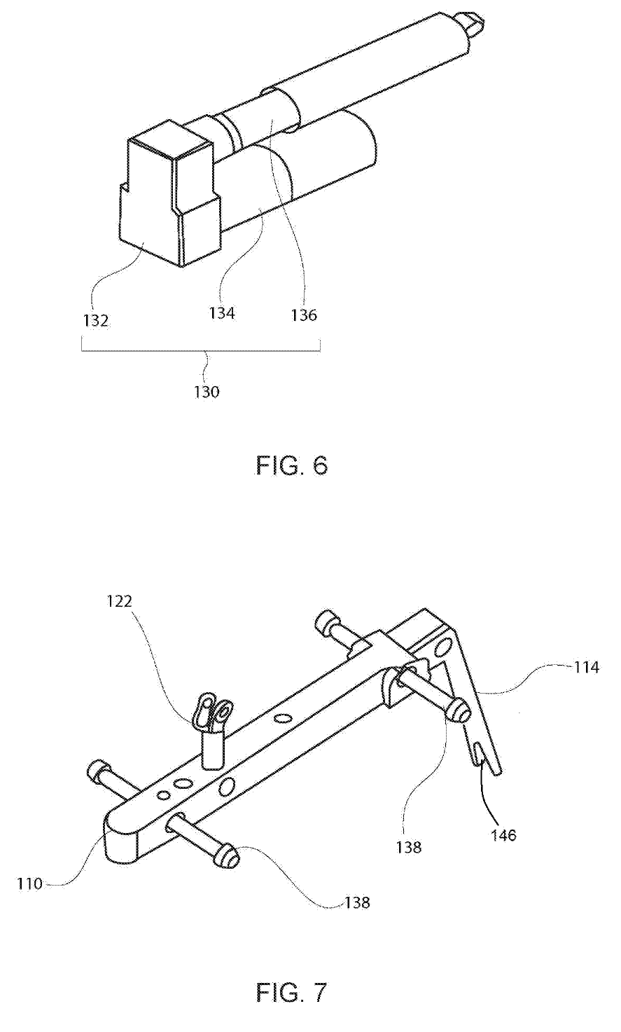

Turning now to the close-up view of FIG. 6, the first plate 102 may be actuated by an electric linear actuator motor 130. The electric linear actuator motor 130 is fully operable through electrical power. The electric linear actuator motor 130 comprises a plurality of gears 132 and a speed control mechanism 134 to manipulate a rod 136. The rod 136 may be linearly extended out to produce a linear force.

FIG. 7 illustrates an exemplary hinged arm 110 that is used to translate the linear motion of the rod 136 to the arced motion of the first plate 102. In one embodiment, the rod 136 operatively connects to the hinged arm 110, and when extended, applies a linear force thereto. The linear force causes the hinged arm 110 to displace the first plate 102 in an arced motion. The hinged arm 110 serves as the link between the electric linear actuator motor 130 and the first plate 102. In this manner, the first plate 102 is induced in an arced translation on and off the second plate 106. In some embodiments, the hinged arm 110 further includes a first link bar 114 configured to pivot the hinged arm 110 about the frame 140. The hinged arm 110 further includes a set of adapter columns 138, which are configured to hingedly mount the hinged arm 110 to the frame 140.

As discussed above, the electric linear actuator motor 130 comprises a rod 136. The electric linear actuator motor 130 is configured to electrically extend and retract the rod 136 in a reciprocating linear motion. The hinged arm 110 operatively joins with the rod 136 of the electric linear actuator motor 130 through an arm adapter 112. A plate adapter 104 operatively connects the hinged arm 110 to the first plate 102, such that the hinged arm 110 translates the linear motion of the rod 136 to the arced motion followed by the first plate 102. In this manner, the arced motion of the hinged arm 110 translates to the arced motion of the first plate 102.

The arced motion of the first plate 102 enables the first plate 102 to press 100 against the second plate 106 at an angle, rather than a vertical pressing action. The angled engagement by the first plate 102 allows both plates 102, 106 to adapt to a variety of sizes and shapes of substrates, such as drinking mugs, garments, trophies, chips, and other variously sized and dimensioned objects.

For example, even among similarly sized coffee cups, size differences occur, for example, among different batches or manufacturing lots. Such size differences may result in variations in the pressure applied by the plates of the heat transfer press to the coffee cup. This can result in too much, or too little, plate pressure which can adversely affect the heat transfer pressing process. In one embodiment, a limit switch 128 controls the pressure and speed of the down stroke of the first plate 102 towards the pressing position.

Looking back at FIG. 1, the press 100 further includes a control portion 120 that automates the linear actuator motor 130 to press and release the plates against each other in predetermined intervals. The control portion 120 comprises a digital temperature and time controller 124 that is configured to set a predetermined temperature and a predetermined duration of time for operation of the press 100. In one embodiment, the control portion 120 actuates the electric linear actuator motor 130 to induce the first plate 102 to the pressing position when the predetermined temperature has been achieved. In another embodiment, the control portion 120 actuates the electric linear actuator motor 130 to induce the first plate 102 to the open position when the predetermined duration of time has expired.

For example, the first plate 102 is actuated to press onto the second plate 106 every thirty seconds, and then to release before repressing again in fifteen seconds, and so on. The control portion 120 may also be used to regulate the temperature and pressure of the plates 102, 106, and to power on and off the press 100. Thus, the control portion 120 reduces the need for manual labor and enables more efficient heat transfer pressing. Both the electric linear actuator motor 130 and the control portion 120 are fully electrical in operation.

As shown in FIG. 3, a display screen 122 enables a user to read the temperature, pressure, and time variables during operation of the press 100. A start switch 126 operatively connects to a power source to power on and off the press 100. An electrical control box 142 provides the various wires and circuitry for regulating the electrical components of the press 100, such as the electric linear actuator motor 130 and the control portion 120.

Thus, the press 100 uses the electric linear actuator motor 130 to control the up and down, open and close of the first plate 102. This allows the press 100 to achieve the best heat transfer effect and avoids the drawbacks of traditional heat press transfer machines, which control the pressure through manual or pneumatic control mode. The press 100 also simplifies the controls and displays through the control portion 120 to reduce operational expenditures for heat transfer pressing.

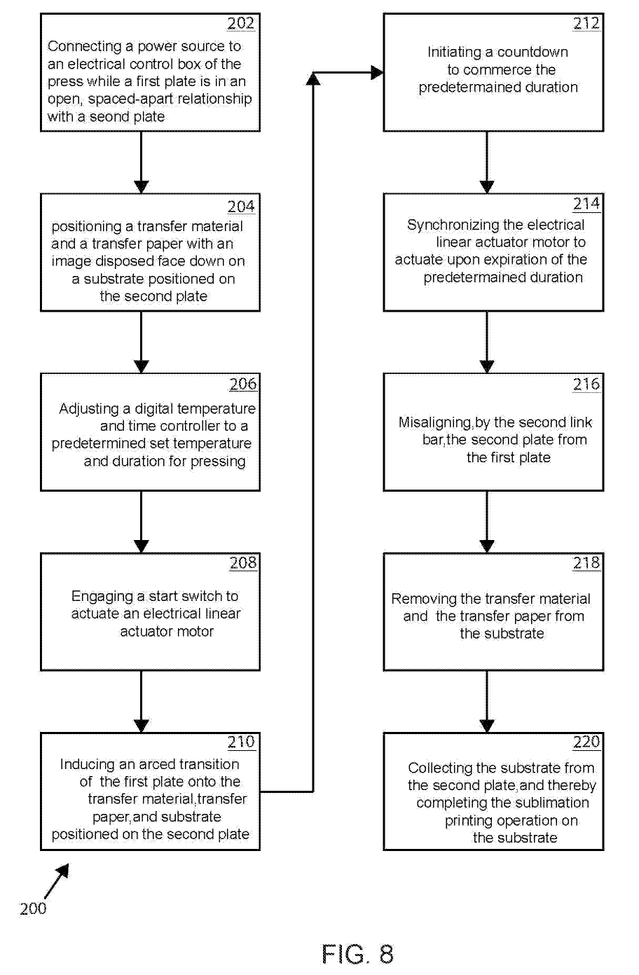

FIG. 8 illustrates a flowchart diagram of an exemplary method 200 for automated heat transfer pressing. The method 200 is configured to enable fully automated, electrical heat transfer pressing between a first plate 102 and a second plate 106 with the first plate 102 moving in an arced motion to enable greater printing options. The method 200 may utilize an electric linear actuator motor 130 that induces an arced translation of the heated first plate 102 onto the fixed second plate 106 to achieve efficient dye sublimation printing. The method 200 also enables control over temperature and pressure through a control portion 120, which automates the linear actuator motor 130 to press and release the plates 102, 106 against each other in predetermined intervals.

The method 200 may include an initial Step 202 of connecting a power source to an electrical control box 142 of the press 100 while a first plate 102 is in an open, spaced-apart relationship with a second plate 106. The electrical control box 142 provides the various wires and circuitry for regulating the electrical components of the press 100, such as the electric linear actuator motor 130. A further Step 204 comprises positioning a transfer material and a transfer paper with an image disposed face down on a substrate positioned on the second plate 106. In this Step 204, the pressure may be adjusted through a limit switch 128. In one possible embodiment, the limit switch 128 enables ten pressure grades for controlling the first plate 102.

The method 200 may also include a Step 206 of adjusting a digital temperature and time controller 124 to a predetermined set temperature and duration for pressing. At this Step 206, the first plate 102 begins to heat up. The control portion 120 comprises a digital temperature and time controller 124 that is configured to set a predetermined temperature and a predetermined duration for operation of the press 100. In one embodiment, the control portion 120 actuates the electric linear actuator motor 130 to induce the first plate 102 to the pressing position when the predetermined temperature has been achieved.

A Step 208 comprises engaging a start switch 126 to actuate an electric linear actuator motor 130. The start switch 126 may be pushed with two hands when the predetermined temperature has been achieved. The start switch 126 operatively connects to a power source to power on and off the press 100. An electrical control box 142 provides the various wires and circuitry for regulating the electrical components of the press 100, such as the electric linear actuator motor 130 and the control portion 120.

A Step 210 may include inducing an arced translation of the first plate 102 onto the transfer material, transfer paper, and substrate that are positioned on the second plate 106. The second plate 106 is automatically aligned with the first plate 102 through a second link bar 116. The arced motion of the first plate 102 enables the first plate 102 to press against the second plate 106 at an angle, rather than a vertical pressing action. The angled engagement between the first plate 102 allows both plates 102, 106 to adapt to a variety of sizes and shapes of substrates, such as drinking mugs, garments, trophies, chips, and other variously shaped and dimensioned objects.

A Step 212 may include initiating a countdown to commence the predetermined duration. The control portion 120 actuates the electric linear actuator motor 130 to induce the first plate 102 to the open position when the predetermined duration has expired. A Step 214 may include synchronizing the electric linear actuator motor 130 to actuate upon expiration of the predetermined duration. Upon termination of the predetermined duration, the electric linear actuator motor 130 lifts the first plate 102 off the second plate 106. The control portion 120 regulates the timer and the electric linear actuator motor 130 to operate in synchronization.

A Step 216 may then include misaligning, by the second link bar 116, the second plate 106 from the first plate 102. In one embodiment, the second plate 106 comprises a pair of draw out slides 108 configured to expand and retract the surface area of the second plate 106. A second link bar 116 is configured to laterally displace the second plate 106 out of alignment with the first plate 102 from the open position. The second link bar 116 laterally displaces the second plate 106 in alignment with the first plate 102 from the pressing position. Thus, the second plate 106 is synchronized to adjust its alignment based on the position of the first plate 102.

A Step 218 comprises removing the transfer material and the transfer paper from the substrate. A final Step 220 comprises collecting the substrate from the second plate 106, and thereby completing the sublimation printing operation on the substrate. The press 100 works to transfer permanent, high-temperature sublimation dyes containing a desired photograph or print, onto substrates, such as ceramic mugs, tiles, trophies, garments, chips, beer steins, and the like. The first plate 102 and the second plate 106 are sufficiently heated and pressed against each other at a sufficient pressure to achieve sublimation and transfer of a dye onto the desired substrate.

Thus in conclusion, an automated heat transfer press 100 and method 200 for automated heat transfer pressing provides an electric linear actuator motor 130 that induces an arced translation of a first plate 102 onto a second plate 106 for dye sublimation printing. The arced motion of the first plate 102 enables the first plate 102 to press against the second plate 106 at an angle, rather than a vertical pressing action; whereby adaption to a variously sized and dimensioned substrates is possible. A control portion 120 regulates the linear actuator motor 130 to press and release the plates 102, 106 against each other in predetermined temperatures and intervals.

Since many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalence.

1. An electrical automated heat press, the press comprising:
a frame;
at least one second plate;
at least one first plate;
a electric linear actuator motor configured to attach to the at least one first plate to move the at least one first plate between an open position, wherein the at least one first plate is moved in an arced motion away from the at least one second plate, and a closed position, wherein the at least one first plate is moved in an arced motion toward, and pressed against the at least one second plate;
a second link bar, the second link bar configured to laterally displace the at least one second plate out of alignment with the at least one first plate from the open position, the second link bar further configured to laterally displace the at least one second plate in alignment with the at least one first plate from the pressing position; and
a control portion to regulate actuation of the electric linear actuator motor.

2. The electrical automated heat press in claim 1, wherein the electric actuator motor comprises a hinged arm configured to operatively join with a rod of the electric linear actuator motor, the rod configured to move in a linear motion.

3. The electrical automated heat press in claim 2, wherein the hinged arm is configured to translate the linear motion of the rod to the arced motion.

4. The electrical automated heat press in claim 3, wherein the hinged arm further includes a first link bar, the first link bar configured to pivot the hinged arm about the frame through a link adapter configured to connect the hinged arm to the at least one first plate.

5. The electrical automated heat press in claim 4, wherein the controller device is configured to set a predetermined duration, whereby the controller actuates the electric linear actuator motor to induce the at least one first plate to the open position when the predetermined duration expires.

6. The electrical automated heat press in claim 1, wherein the controller device further comprises a limit switch, the limit switch configured to control the speed and pressure of the at least one first plate while moving towards the at least one second plate.

7. The electrical automated heat press in claim 1, wherein the at least one first plate comprises a heated device, the heated device configured to generate heat uniformly through the first plate.

8. An electrical automated heat press, the press comprising:
a frame, the frame configured in a fixed position for supporting the press;
at least one second plate, the at least one second plate configured to join with the frame;
at least one first plate, the at least one first plate configured to controllably generate heat, the at least one first plate further configured to reciprocally move in an arced motion towards and away from the at least one second plate,
whereby the at least one first plate is disposed in a spaced-apart relationship to the at least one second plate from an open position,
whereby the at least one first plate is disposed in an engaged, substantially aligned relationship with the at least one second plate from a pressing position;
a second link bar, the second link bar configured to laterally displace the at least one second plate out of alignment with the at least one first plate from the open position, the second link bar further configured to laterally displace the at least one second plate in alignment with the at least one first plate from the pressing position;
an electric linear actuator motor, the electric linear actuator motor comprising a rod, the electric linear actuator motor configured to extend and retract the rod in a linear motion;
a hinged arm, the hinged arm configured to operatively join with the rod of the electric linear actuator motor, the hinged arm further configured to translate the linear motion of the rod to the arced motion;
a plate adapter, the plate adapter configured to operatively connect the hinged arm to the at least one first plate,
whereby the arced motion of the hinged arm translates to the arced motion of the at least one first plate; and
a control portion, the control portion configured to regulate actuation of the electric linear actuator motor, the control portion further configured to set a predetermined temperature and a predetermined duration,
whereby the control portion actuates the electric linear actuator motor to induce the at least one first plate to the pressing position when the predetermined temperature has been achieved,
whereby the control portion actuates the electric linear actuator motor to induce the at least one first plate to the open position when the predetermined duration has expired.

9. The press of claim 8, wherein the at least one second plate further comprises a pair of draw out slides, the pair of draw out slides configured to expand and retract the surface area of the at least one second plate.

10. The press of claim 8, wherein the electric linear actuator motor further comprises a plurality of gears and a speed control mechanism, the plurality of gears and a speed control mechanism configured to control displacement of the rod.

11. The press of claim 8, wherein the hinged arm further includes an arm adapter, the arm adapter configured to operatively connect the hinged arm to the electric linear actuator motor.

12. The press of claim 8, wherein the hinged arm further includes a first link bar, the first link bar configured to pivot the hinged arm about the frame.

13. The press of claim 8, wherein the hinged arm further includes a set of adapter columns, the set of adapter columns configured to hingedly mount the hinged arm to the frame.

14. The press of claim 8, further including a housing, the housing configured to at least partially cover the hinged arm and the electric linear actuator motor.

15. The press of claim 8, wherein the control portion comprises a digital temperature and time controller, the digital temperature and time controller configured to set a predetermined temperature and a predetermined duration for operation of the press.

16. The press of claim 8, wherein the control portion comprises a display screen for displaying the predetermined temperature, the predetermined duration, and a pressure of the at least one first plate.

17. The press of claim 8, further including an electrical control box, the electrical control box configured to regulate the electric linear actuator motor and the control portion.

18. The press of claim 8, further including a start switch, the start switch operatively connected to a power source, the start switch configured to power on and off the press.

19. The press of claim 8, further including a limit switch, the limit switch configured to control the speed and pressure of the at least one first plate while moving towards the at least one second plate.

20. A method for automated heat transfer pressing, the method comprising:
connecting a power source to an electrical control box of the press while at least one first plate is in an open, spaced-apart relationship with at least one second plate;
positioning a transfer material and a transfer paper with an image disposed face up or face down on a substrate positioned on the at least one second plate;
adjusting a digital temperature and time controller to a predetermined set temperature and duration for pressing;
engaging a start switch to actuate an electric linear actuator motor;
inducing an arced translation of the at least one first plate onto the transfer material, transfer paper, and substrate positioned on the at least one second plate;
initiating a countdown to commence the predetermined duration;
synchronizing the electric linear actuator motor to actuate upon expiration of the predetermined duration;
misaligning, by the second link bar, the second plate from the at least one first plate;
removing the transfer material and the transfer paper from the substrate; and
collecting the substrate from the at least one second plate, and thereby completing the sublimation printing operation on the substrate.

21. An electrical automated heat press, the press consisting of:
a frame, the frame configured in a fixed position for supporting the press;
at least one second plate, the at least one second plate configured to join with the frame, the at least one second plate comprising a pair of draw out slides, the pair of draw out slides configured to expand and retract the surface area of the at least one second plate;
at least one first plate, the at least one first plate configured to controllably generate heat, the at least one first plate further configured to reciprocally move in an arced motion towards and away from the at least one second plate,
whereby the at least one first plate is disposed in a spaced-apart relationship to the at least one second plate from an open position,
whereby the at least one first plate is disposed in an engaged, substantially aligned relationship with the at least one second plate from a pressing position;
a heating pipe embedded in the first plate, the heating pipe configured to carry thermal energy uniformly through a core of the first plate;
a second link bar, the second link bar configured to laterally displace the at least one second plate out of alignment with the at least one first plate from the open position, the second link bar further configured to laterally displace the at least one second plate in alignment with the at least one first plate from the pressing position;
an electric linear actuator motor, the electric linear actuator motor comprising a rod, the electric linear actuator motor configured to extend and retract the rod in a linear motion, the electric linear actuator motor further comprising a plurality of gears and a speed control mechanism, the plurality of gears and the speed control mechanism configured to control displacement of the rod;
a hinged arm, the hinged arm configured to operatively join with the rod of the electric linear actuator motor, the hinged arm further configured to translate the linear motion of the rod to the arced motion;
an arm adapter configured to operatively connect the hinged arm to the electric linear actuator motor;
a set of adapter columns configured to hingedly mount the hinged arm to the frame;
a housing configured to at least partially cover the hinged arm and the electric linear actuator motor;
a plate adapter, the plate adapter configured to operatively connect the hinged arm to the at least one first plate,
whereby the arced motion of the hinged arm translates to the arced motion of the at least one first plate;
a control portion, the control portion configured to regulate actuation of the electric linear actuator motor, the control portion further configured to set a predetermined temperature and a predetermined duration, wherein the control portion comprises a digital temperature and time controller, the digital temperature and time controller configured to set a predetermined temperature and a predetermined duration for operation of the press,
whereby the control portion actuates the electric linear actuator motor to induce the at least one first plate to the pressing position when the predetermined temperature has been achieved,
whereby the control portion actuates the electric linear actuator motor to induce the at least one first plate to the open position when the predetermined duration has expired;
an electrical control box, the electrical control box configured to regulate the electric linear actuator motor and the control portion;
a start switch operatively connected to a power source, the start switch configured to power on and off the press; and
a limit switch configured to control the speed and pressure of the at least one first plate while moving towards the at least one second plate.
